(19) United States
(12) Patent Application Publication
(10) Pub. No.: US 2016/0262824 A1
(43) Pub. Date: Sep. 15, 2016

(54) LOOP ELECTRODE

(71) Applicant: TROKAMED GMBH, Gelsingen (DE)

(72) Inventor: Karlheinz Troendle, Geisingen (DE)

(73) Assignee: TROKAMED GMBH, Geisingen (DE)

(21) Appl. No.: 14/442,136

(22) PCT Filed: Nov. 7, 2013
(86) PCT No.: PCT/EP2013/073313

§ 371 Date: May 12, 2015

(30) Foreign Application Priority Data
Nov. 13, 2012 (DE) 10 2012 110 894.3
Dec. 3, 2012 (DE) 10 2012 111 736.5

(51) Int. Cl. A61B 18/14

(52) U.S. Cl. CPC A61B 18/14; A61B 2018/141; A61B 2017/0046

(57) ABSTRACT

A loop electrode comprising a shaft and handle associated therewith, the shaft consisting of an inner shaft (2) and an outer shaft (3). The inner shaft (2) is permanently connected to the handle and the outer shaft (3) can be displaced on the inner shaft (2). The inner shaft (2) has a loop (6) comprising a first loop head (8) and a second loop head, the loop (6) comprising the first loop head (8) that can be decoupled from the inner shaft (2).

### BACKGROUND OF THE INVENTION

The invention relates to a loop electrode.

Various loop electrodes are known from the prior art and are in common use. There are principally two different types of loop electrodes. For example, a loop electrode is known in which the two wire ends of the loop are connected permanently, i.e. in one piece, to an inner shaft. A disadvantage of this, however, is that the instrument can be used only in the closed state. This can lead to complications if the anatomical conditions are unfavorable.

Furthermore, the prior art discloses a loop electrode in which both wire ends are releasable on the inner shaft. Again, a disadvantage here is that this type of loop electrode can lead to difficulties in the hands of unpracticed users, since handling has proven complicated.

The problem addressed by the invention is to make available a loop electrode that overcomes the disadvantages of the prior art and, in so doing, to offer a solution that is extremely user friendly and can be applied quickly.

### SUMMARY OF THE INVENTION

The foregoing objects are achieved by the loop electrode of the present invention.

A loop electrode according to the invention is composed of a shaft and of an associated handle. The shaft in turn is composed of an inner shaft and of an outer shaft. The inner shaft is permanently connected to the handle. The outer shaft is movable on the inner shaft. This mobility results primarily from the fact that the outer shaft has a larger diameter than the inner shaft. In addition, the outer shaft is not connected to the inner shaft. Moreover, the inner shaft has a loop. This loop serves, for example, for the coagulation of human tissue.

The loop electrode according to the invention is preferably used in the field of hysterectomy and employs a monopolar or bipolar technique. The tissue is separated by means of the inner shaft being pulled through the outer shaft in the direction of the proximal end. By means of this procedure, the loop at the distal end is contracted until it is located partially or almost completely in the outer shaft and the tissue is separated.

The loop electrode according to the invention is designed in such a way that the loop comprises exactly one first loop head that can be decoupled from the inner shaft. In one illustrative embodiment, this first loop head can be designed as a ball. However, it is also possible in principle that the first loop head is shaped as a cube, pyramid, cylinder, oval ball or has another shape that exceeds the diameter of the wire.

The second loop head is permanently connected. That is to say, the second loop head cannot be released from the inner shaft, as is the case with the first loop head for example. This also means that decoupling and coupling are not possible. Instead, the second loop head remains connected to the inner shaft even when the outer shaft has not been pulled onto the inner shaft.

The fact that the loop can be decoupled from the inner shaft only at one end advantageously means that the person using the loop electrode can also take unfavorable anatomical conditions into account during the operating procedure.

With the loop pushed out, the outer shaft can be placed in two positions with respect to the inner shaft. This serves to allow the loop to be used, on the one hand, as a closed system and, on the other hand, as a half-open system. In this way, the user is afforded greater flexibility. Thus, depending on the anatomical circumstances and on the amount of tissue, the user chooses whether it is advantageous to trap the tissue or encircle the tissue. For example, when there is a large amount of tissue, he decouples the loop head from the inner shaft and places the loop around the body tissue that is to be removed. By virtue of the fact that the second loop head is connected permanently to the inner shaft, the person using the loop electrode can advantageously use the inner shaft to control where the loop electrode is guided. He can then couple the first loop head to the inner shaft again. For particular protection of this advantageous solution is also the coupling itself, which can be designed as a kind of collecting pot.

Moreover, a loop according to the invention is designed such that it can be pulled into and pushed out of the outer shaft. In principle, the loop electrode according to the invention can be used in two operating modes. On the one hand, it can be used as a half-open system. In this half-open system, the loop is permanently connected to the inner shaft at one end, and the second end can be connected to the inner shaft by means of an auxiliary instrument, for example forceps. A ball, for example, is mounted on the wire at the second loose end, which ball is fitted into the inner shaft in order to close the loop. The ball is locked automatically in the inner shaft by the spring action of the wire. As soon as the outer shaft closes the area into which the ball is fitted, the ball is secured and can no longer come loose.

The second operating mode is a closed system. In this variant, the outer shaft cannot be pulled onto the area of the collecting pot of the inner shaft. This ensures that the ball cannot come loose from the inner shaft. This is achieved by a securing means which, for example, can be a ring, wherein the ring in a preferred illustrative embodiment can be clipped onto and/or unclipped from the inner shaft. In the same way, the securing means in the form of the ring can also be directly clipped onto and/or unclipped from the proximal end of the outer shaft. In this way, the loop electrode is secured for use at any time.

Moreover, the outer shaft has a finger recess. This finger recess serves for better control of the loop electrode, since a relative movement of the outer shaft with respect to the inner shaft can be executed by interaction with the finger depression arranged on the outer shaft and the handle mounted permanently on the inner shaft.

The inner shaft has a coupling. This coupling is suitable for receiving and releasing the first loop head. The shape of the coupling adapts to the shape of the first loop head. The coupling is intended to produce a force-fit connection between the first loop head and the inner shaft, although this connection is also intended to be easily undone. Particularly preferably, the coupling is designed as a collecting pot. This advantageously permits simple control by the user, since the first loop head can be connected to the inner shaft without any great effort and without special training. The particular shape of the coupling allows the loop head to be easily removed and, when reinserted, the loop head is caught in the collecting pot and is thereby self-locking. The self-locking is achieved by the fact that the ball is enclosed in the collecting pot and, by the spring action of the wire, is pulled forward into the collecting pot and is thus automatically held.

In addition, the loop is partially enclosed by plastic. This serves to ensure that only defined areas can be coagulated or cut. Therefore, it is not the whole loop that is enclosed by plastic, but instead only the part thereof that is not intended to serve for cutting. In a preferred illustrative embodiment, the loop is designed for example as a square, in which one of the corner areas is designed as a distal end, for example without a plastic cover. The opposite end is preferably directly connected or connectable proximally to the inner shaft.

In another illustrative embodiment, the loop can also have a hexagonal shape. The advantage of this design is that the opened loop has a greater surface and, as a result, a larger amount of tissue can be enclosed. A further advantage is that, upon removal of small tissues, e.g. of stalked polyps, the loop can be pulled in as far as the first corners, resulting then in the formation of a small loop. Adaptations of the shape and length are left to the discretion of a person skilled in the art, as long as the usability of the hexagonal loop is ensured.

Within the context of the application, the terms “proximal” and “distal” are to be understood from the point of view of the user.

The half-open position is an expression signifying that the first loop head is withdrawn from the collecting pot. By contrast, the closed position of the loop signifies that the first loop head is located in the collecting pot and the outer shaft has been pulled over the collecting pot for securing it.

In a preferred illustrative embodiment, the coupling has the collecting pot, where the collecting pot forms a conical recess. The collecting pot is designed in such a way that, with the aid of the wire, the loop head is held with self-locking in the collecting pot. The coupling is of tubular design, wherein the collecting pot is formed by two distally located tabs.

### BRIEF DESCRIPTION OF THE DRAWINGS

Further advantages, features and details of the invention will become clear from the following description of a preferred illustrative embodiment. Details are to be found in the figures, in which:

FIG. 1 shows a side view of a loop electrode according to the invention in the state ready for use;

FIG. 2 shows a side view of the loop electrode according to FIG. 1, in the state not available for use.

FIG. 3 shows an enlarged view of the loop electrode according to the invention from FIG. 1 or 2;

FIG. 4 shows an enlarged view of the loop electrode according to the invention from FIG. 1 or 2;

FIG. 5 shows a loop with a hexagonal shape;

FIG. 5.1 shows the loop according to FIG. 5, wherein the hexagonal loop is shown having been pulled in by approximately half;

FIG. 6 shows a coupling according to the invention with collecting pot.

### DETAILED DESCRIPTION

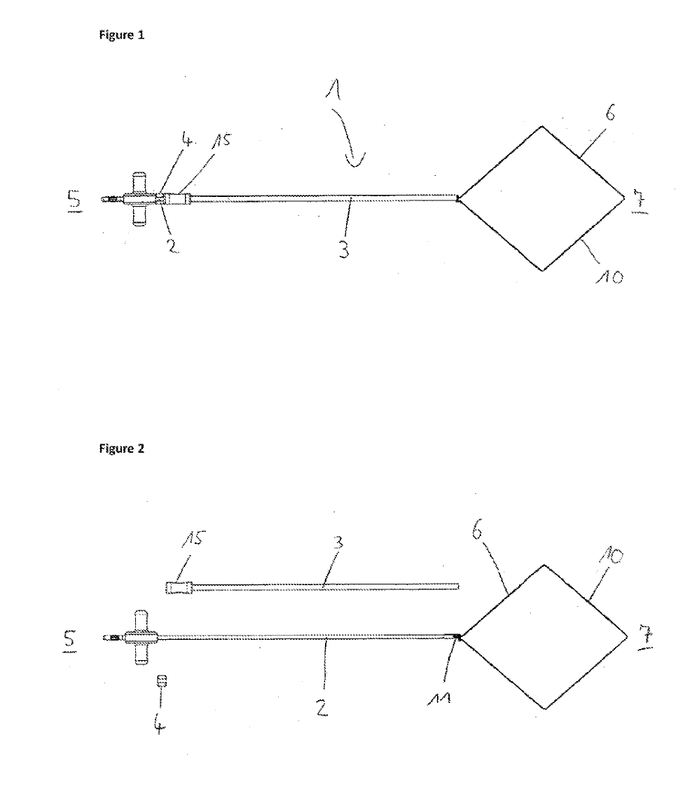

It will be seen from FIG. 1 how a loop electrode 1 has a loop 6 at its distal end 7. The loop 6 is composed of a wire 10 which, in FIG. 1, has a square shape. FIG. 1 also shows a securing ring 4 at the proximal end 5 of the loop electrode 1, which securing ring is clipped onto the inner shaft 2. In addition, an outer shaft 3 is shown, which comprises a finger recess 15. A handle (not shown in any detail) can be mounted at the proximal end 5 of the loop electrode 1. The handle is then permanently connected to the inner shaft 2, which means that the handle predefines the movements of the inner shaft 2.

It will further be seen from FIG. 2 that the inner shaft 2 has a smaller diameter than the outer shaft 3. The figure also shows how the securing ring 4 can be unclipped from the inner shaft 2.

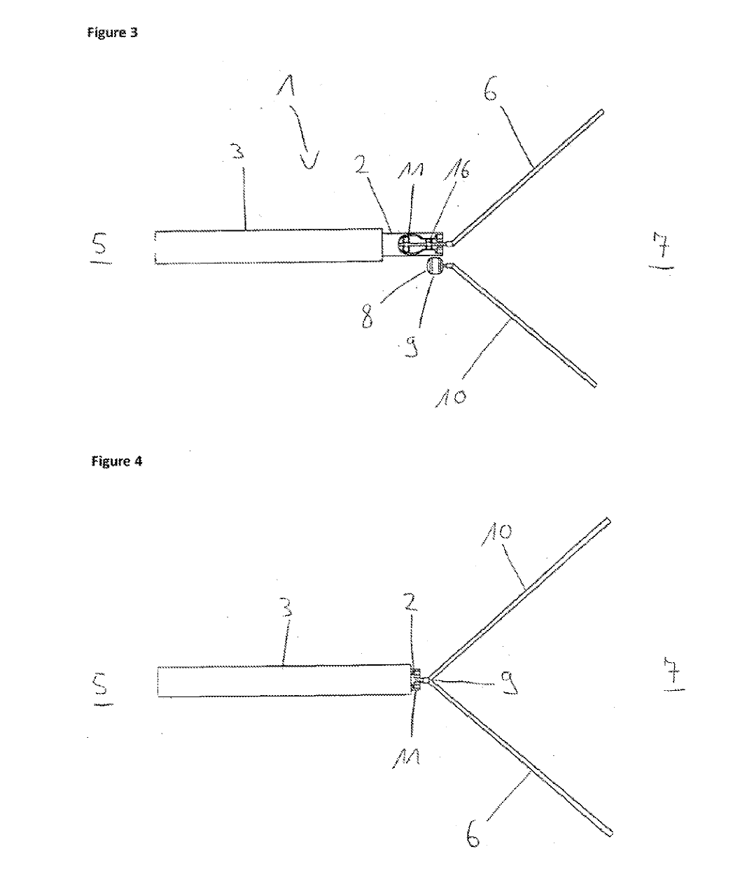

FIG. 3 shows an enlarged detailed view of part of the loop electrode 1 from FIG. 1. It shows how the wire 10 of the loop 6 forms a first loop head 8 in the shape of a ball 9. In addition, a coupling 11 is shown which permits a simple insertion of the ball 9, since the coupling is designed in the form of a conical head 16. That is to say, the ball 9 can be easily fitted into the provided hole of the coupling 11, wherein the wire 10 is guided through a channel connected to the hole in order to secure the ball 9 in the coupling 11.

Thereafter, as is shown in FIG. 4, the outer shaft 3 is then guided over the inner shaft 2 and thus over the coupling 11 and also the ball 9. When the outer shaft 3 is pushed farther toward the distal end 7, the wire is then folded together and pulled into the interior of the outer shaft 3. This procedure can be easily revised by pulling the outer shaft 3 in the proximal direction 5.

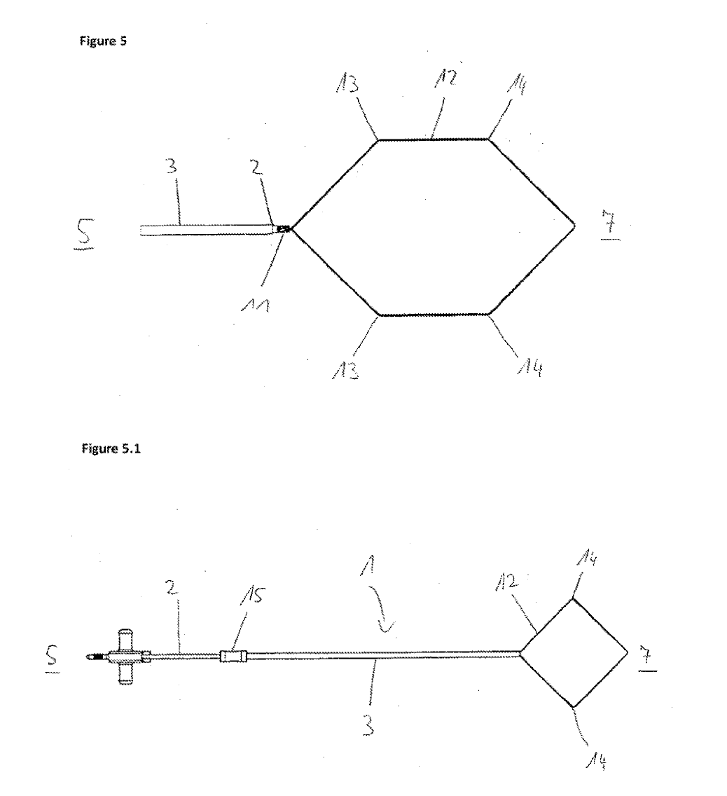

In FIG. 5, a hexagonal wire 12 is shown with the central corners 13, wherein the corners 13 are arranged proximally, and the front corners 14, which are arranged distally.

In FIG. 5.1, the hexagonal wire 12 has been pulled back by about half, which means here that it has been pulled back in the proximal direction. The front corners 14 now form a small loop.

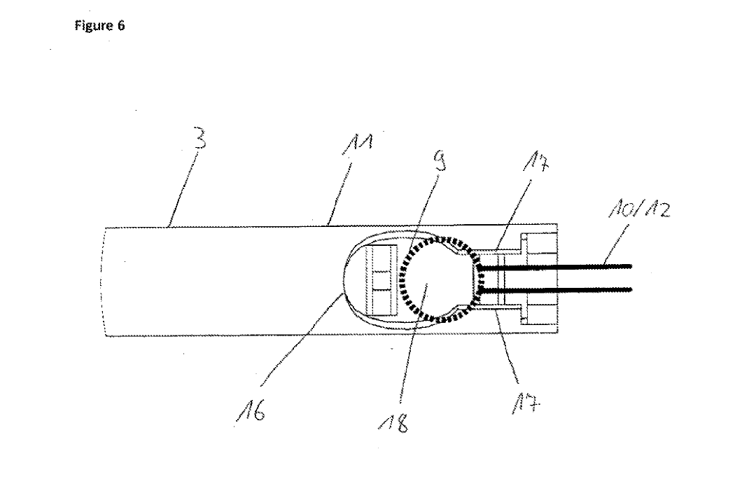

In FIG. 6, the inner shaft 3 with the coupling 11 is shown in an enlarged form. The figure shows a cavity 16 (conical head), for receiving the ball 9, and also the drawn-in tabs 17 acting as a depth stop for the ball 9. The collecting pot 18 moreover serves for the fixing and self-locking of the ball 9. The broken line indicates the ball 9 that is held in the collecting pot 18.

1. A loop electrode comprising a shaft and an associated handle, the shaft comprises an inner shaft (2) and an outer shaft (3), the inner shaft (2) is connected to the handle and the outer shaft (3) is movable on the inner shaft (2), the inner shaft (2) comprises a loop (6) with a first loop head (8) and a second loop head, wherein the first loop head (8) is selectively decoupled from the inner shaft (2).

2. The loop electrode as claimed in claim 1, wherein the first loop head (8) is a ball (9).

3. The loop electrode as claimed in claim 1, wherein the loop (6) is adapted to move into and/or out of the outer shaft (3).

4. The loop electrode as claimed in claim 2, further including a securing ring (4) adapted to be clipped onto and/or unclipped from the inner shaft (2) for adjusting between a half-open position and a closed position of the loop.

5. The loop electrode as claimed in claim 4, wherein a further securing ring (4) is clipped onto and/or unclipped from the outer shaft (3).

6. The loop electrode as claimed in claim 2, wherein the outer shaft (3) has a finger recess (12).

7. The loop electrode as claimed in claim 2, wherein the inner shaft (2) has a coupling (11) adapted to receive the first loop head (8).

8. The loop electrode as claimed in claim 7, wherein the coupling (11) comprises a collecting pot (18).

9. The loop electrode as claimed in claim 2, wherein the loop (6) is partially enclosed by plastic.
